(19) United States
(12) Patent Application Publication
(10) Pub. No.: US 2026/0069878 A1
(43) Pub. Date: Mar. 12, 2026

(54) BIO-FUNCTION ACTIVATION DEVICE AND METHOD THEREOF

(71) Applicants: Jeoung Gi PARK, Yongin-si Gyeonnggi-do (KR); Chang Ho KIM, Goyang-si (KR); Jin Han JUNG, Gwangmyeong-si (KR); Juventa Science, Seoul (KR)

(72) Inventors: Jeoung Gi PARK, Yongin-si Gyeonnggi-do (KR); Chang Ho KIM, Goyang-si (KR); Jin Han JUNG, Gwangmyeong-si (KR)

(21) Appl. No.: 19/218,056

(22) Filed: May 23, 2025

(30) Foreign Application Priority Data
Sep. 11, 2024 (KR) 10-2024-0123995

(51) Int. Cl. A61N 1/40

(52) U.S. Cl. CPC A61N 1/40

(57) ABSTRACT

A bio-function activation device includes an oscillator that outputs an electric signal of a specific frequency, modulates an electromagnetic wave according to the electric signal of the specific frequency to output a longitudinal wave corresponding to the specific frequency, wherein the specific frequency is a frequency determined based on a body rhythm when a physical condition of a user is in a specific state in order to change the physical condition of the user to the specific state.

### CROSS-REFERENCE TO RELATED APPLICATION

This application claims the benefit of priority under 35 U.S.C. § 119 (a) to Korean Patent Application No. 10-2024-0123995 filed in the Korean Intellectual Property Office on Sep. 11, 2024, the entire contents of which are incorporated herein by reference.

### BACKGROUND

### (a) Technical Field

The present invention relates to a bio-function activation device for changing a physical condition using modulated electromagnetic waves and a method thereof.

### (b) Background Art

A bio-function activation technology is a technology that conceptually evaluates the health status of a living organism, promotes homeostasis of the body, and activates bio-metabolism. This bio-function activation technology is divided into a pharmacological material approach and an energy-based approach. Among these, the pharmacological material approach technology is a method to find out how substances interact to create functional changes in living organisms. Generally, pharmacological substances target natural products and contribute to disease treatment or bio-metabolism activation through pharmacological mechanisms. Meanwhile, the energy-based technology is a method to activate a bio-function by normalizing the energy imbalance of a living organism, and is being studied as energy medicine, a category of traditional oriental medicine or alternative/complementary medicine in the West.

Recently, research on energy medicine is actively being conducted, going beyond the stage of viewing and approaching a human body as a mechanical and material entity and viewing the human body as an energetic entity and diagnosing and treating the human body through energy fields. The energy sources include electric fields, magnetic fields, electromagnetic waves, and longitudinal waves.

In particular, the longitudinal wave is a technology that provides many clues for the development of energy medicine, and devices for therapeutic purposes using the longitudinal wave are continuously being developed overseas.

However, the method of generating the longitudinal waves has not deviated from the initial method of using coils or plasma. For example, there are methods of generating the longitudinal waves by using a special coil to cancel out electromagnetic waves using two coils wound in opposite directions or by canceling the electromagnetic waves in a geometric shape to emit the non-hertzian waves, and by generating resonant waves to generate the longitudinal waves.

The longitudinal wave generator configured in this way has limitations in terms of mass production, price, marketability, usability, and application fields due to the large size of the generator.

### SUMMARY OF THE DISCLOSURE

The present invention is intended to solve the problems of the above-mentioned related art, and an object thereof is to provide a bio-function activation device and method thereof capable of generating longitudinal waves with a simple configuration by using an electric signal oscillator manufactured with a semiconductor chip.

In order to achieve the above-mentioned object, according to one embodiment of the present invention, there is provided a bio-function activation device, which comprises an oscillator that outputs an electric signal of a specific frequency, and modulates an electromagnetic wave according to the electric signal of the specific frequency to output a longitudinal wave corresponding to the specific frequency, wherein the specific frequency is a frequency determined based on a body rhythm when a physical condition of a user is in a specific state in order to change the physical condition of the user to the specific state.

According to another aspect of the present invention, there is provided a bio-function activation device including: a battery that supplies power; an oscillator that is electrically connected to the battery and outputs an electric signal of a specific frequency when power is applied from the battery; a modulation module that is electrically connected to the oscillator, receives the electric signal of the specific frequency output from the oscillator, modulates an electromagnetic wave generated by the electric signal of the specific frequency, and outputs an electric signal in which the electromagnetic wave is modulated; and an output module that is electrically connected to the modulation module, receives an electric signal output from the modulation module, and outputs the modulated electromagnetic wave according to the received electric signal to an external space.

According to yet another aspect of the present invention, there is provided a bio-function activation method for changing a physical condition of a user to a specific state, the bio-function activation method including the steps of: determining a frequency of an electric signal based on a body rhythm when the physical condition of the user is in a specific state; outputting an electric signal according to the determined frequency; modulating an electromagnetic wave generated by the output electric signal into a longitudinal wave; and outputting the modulated longitudinal wave to an external space, wherein the electric signal is a square wave of positive voltage, and frequencies of the electromagnetic wave generated by the outputted electric signal and the modulated longitudinal wave output to an external space are determined by the determined frequency.

According to the present invention, the bio-function activation device capable of generating the longitudinal wave with a simple configuration is provided by using the electric signal oscillator manufactured with the semiconductor chip, thereby resolving limitations in terms of existing mass production, price, marketability, usability, and fields of application.

In addition, according to the present invention, by providing each individual with a wearable, small-sized bio-function activation device, each individual can activate the bio-function, maintain a positive physical condition, and promote health.

### BRIEF DESCRIPTION OF THE DRAWINGS

FIG. 1 is a block diagram illustrating a schematic configuration of a bio-function activation device according to the present invention.

FIG. 2 is a photograph illustrating an actually manufactured product of the bio-function activation device according to the present invention.

FIG. 3 is a diagram schematically illustrating a configuration of a modulation module according to the present invention.

FIG. 4 is a flowchart illustrating a bio-function activation method for changing a physical condition of a user to a specific state according to the present invention.

### DETAILED DESCRIPTION

The present invention may have various modifications and various embodiments, and specific embodiments are illustrated in the drawings and described in detail in the detailed description. However, this is not intended to limit the present invention to specific embodiments, but should be understood to include all modifications, equivalents, or substitutes included in the spirit and technical scope of the present invention.

The terminology used in this specification is only for describing particular embodiments and is not intended to limit the present invention. The singular expression includes the plural expression unless the context clearly indicates otherwise. As used herein, the terms “comprise” or “have” and the like are intended to specify the presence of a feature, number, step, operation, component, part or combination thereof described in the specification, but should be understood to not exclude in advance the possibility of the presence or addition of one or more other features, numbers, steps, operations, components, parts or combinations thereof.

In addition, the components of the embodiments described with reference to each drawing are not limited to the embodiments described herein, and may be implemented to be included in other embodiments within the scope in which the technical spirit of the present invention is maintained, and it goes without saying that multiple embodiments may also be reimplemented as one integrated embodiment even if a separate description is omitted.

In addition, when describing with reference to the attached drawings, regardless of the drawing symbols, identical components are given identical or related reference symbols, and redundant descriptions thereof are omitted. When describing the present invention, if it is determined that a detailed description of a related known technology may unnecessarily obscure the gist of the present invention, the detailed description thereof is omitted.

Maxwell, who established the foundation of electromagnetism, initially proposed 20 equations, and about 20 years later, Oliver Heaviside integrated them into four, in the process of which the longitudinal electric wave and electric potential terms disappeared, and Maxwell's equations (or Maxwell-Heaviside equations) were completed, with the electric field E equation and the magnetic field B equation as the core concepts. These are Gauss's law, Gauss's magnetic law, Faraday's law, and Ampere's law.

However, Maxwell's equations, although they are empirical theories that form the basis of modern science, have faced many challenges today. Prof. Dr. Konstantin Meyl of Germany pointed out that many of the basic equations of modern electromagnetism were omitted and organized, making them fewer universal equations that only apply in certain situations. In this extension, Prof. Dr. Konstantin Meyl pointed out that electromagnetic waves propagating through space are longitudinal waves in addition to transverse waves.

That is, electromagnetic waves are divided into a Hertz's transverse wave and a Tesla's longitudinal wave. Specifically, they are Hertz's electromagnetic wave (transverse wave), Tesla's longitudinal scalar waves, and Konstantin Meyl's longitudinal magnetic waves. The longitudinal wave is formed when either an electric field or a magnetic field is oriented in the same direction as the direction of propagation. For example, an antenna that forms an electric dipole emits electromagnetic waves that include longitudinal elements, which are also called field vortexes. Furthermore, in a situation where electromagnetic waves are emitted, the longitudinal waves may be formed in the form of a near field within half a wavelength or one wavelength.

The longitudinal waves are waves in which the direction of wave propagation is parallel to the direction of vibration of the medium. Unlike transverse waves that have a negative effect on living organisms, the longitudinal waves may have a positive effect on living organisms. Several studies have illustrated that longitudinal waves have a positive effect on living organisms. For example, it has been found that longitudinal electromagnetic waves can cause an increase in energy (measured by an increase in ATP) in plant mitochondria (Heide Schnabl, Hermann Schnable, Konstantin Meyl, Anti-Aging by Longitudinal Magnetic Waves: A New Approach by Modulating ATP-Levels, OAT (Open Access Text), 2019). The mitochondria exist not only in plant cells but also in animal and human cells, and their product, ATP, is the same and is an indispensable component for all life processes.

The present invention relates to a bio-function activation device that can improve a bio-function of a user by generating a longitudinal wave using an electric signal oscillator manufactured with a semiconductor chip.

The bio-function activation device according to the present invention includes an oscillator that outputs an electric signal of a specific frequency, modulates an electromagnetic wave according to the electric signal of the specific frequency, and outputs a longitudinal wave corresponding to the specific frequency. In this case, the specific frequency is a frequency determined based on the body rhythm when the physical condition of the user is in the specific state in order to change the physical condition of the user into a specific state.

Here, the physical condition of the user being in the specific state may be, for example, a state in which muscle cells are activated, a state in which blood flow in the body increases, a state in which stress or fatigue is relieved, or the like. This specific state may be a physical condition that the user wants to reach from the current physical condition. For example, when the user is currently in a state of high stress or high fatigue, the user may want to be in a state of low stress or fatigue.

In addition, the body rhythm when the physical condition of the user is in the specific state refers to the rhythm of the electromagnetic energy emitted from the body when the human physical condition is in the specific state, or the rhythm, electromagnetic or acoustic stimulation applied to the human body to change the human physical condition to a specific state.

Unlike other longitudinal wave generators, the present invention can be configured to be smaller and simpler by using an electric signal oscillator manufactured with a semiconductor chip.

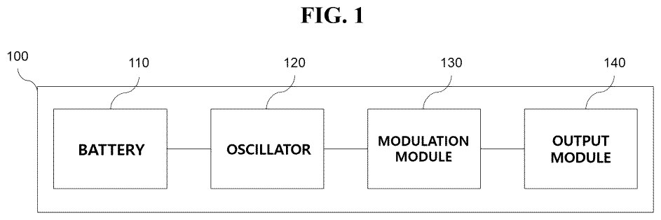

FIG. 1 is a block diagram illustrating a schematic configuration of a bio-function activation device according to the present invention.

Referring to FIG. 1, a bio-function activation device 100 according to the present invention includes a battery 110, an oscillator 120, a modulation module 130, and an output module 140.

First, the battery 110 supplies power. The power supplied from the battery 110 may be a DC (direct current) power source.

The oscillator 120 is electrically connected to the battery 110, and outputs an electric signal of a specific frequency when power is applied from the battery 110. Here, the electric signal output by the oscillator 120 is an AC (alternating current) signal, and in particular, it is preferable that the electric signal is a square wave of positive voltage. In addition, the specific frequency of the electric signal may be a frequency that is preset before the user uses the bio-function activation device 100.

The modulation module 130 is electrically connected to the oscillator 120, receives the electric signal of the specific frequency output from the oscillator 120, modulates the electromagnetic wave generated by the electric signal of the specific frequency, and outputs the electric signal in which the electromagnetic wave is modulated.

The modulation module 130 modulates the electromagnetic wave generated by an electric signal by changing the spin of an electron moving in a wire. In general, the electromagnetic wave generated by the electric signal output from the oscillator 120 is a transverse wave, and the modulation module 130 may modulate the electromagnetic wave into a longitudinal wave and output the electric signal in which the electromagnetic wave is modulated.

The electromagnetic waves resulting from the electric signal output from the modulation module 130, the electric signal passing through the wire between the modulation module 130 and the output module 140, and the electric signal transmitted to the output module 140 are electromagnetic waves modulated by the modulation module 130, and the electromagnetic waves resulting from the electric signal output from the modulation module 130, the electric signal passing through the wire between the modulation module 130 and the output module 140, and the electric signal transmitted to the output module 140 are output to the external space, so that the user using the bio-function activation device 100 may be exposed to these electromagnetic waves.

The output module 140 is electrically connected to the modulation module 130, receives an electric signal output from the modulation module 130, and outputs the electromagnetic wave according to the received electric signal to the external space.

Finally, when power is applied to the oscillator 120, the oscillator 120 outputs the electric signal of the specific frequency, and the output electric signal is modulated into the electromagnetic wave in the modulation module 130 and output as a modulated electromagnetic wave to the external space through the output module 140.

The modulated electromagnetic waves are incident on the user wearing the bio-function activation device 100, and the incident electromagnetic waves stimulate cells constituting the body of the user, particularly centered on the location where the bio-function activation device 100 is worn, and when the user is continuously exposed to the stimulation of the electromagnetic waves, the body of the user changes into the physical condition different from before wearing the bio-function activation device 100.

Meanwhile, when the modulation module 130 modulates the electromagnetic wave by the electric signal from the transverse wave to the longitudinal wave, the frequency of the transverse wave by the electric signal or the longitudinal wave output from the modulation module 130 is determined by the frequency of the electric signal input to the modulation module 130, which is ultimately determined by the frequency of the electric signal output from the oscillator 120.

The bio-function activation device 100 is intended to stimulate the cells of the body to change the physical condition of the user to the specific state, and it is preferable that the specific frequency of the electric signal output from the oscillator 120 is a low frequency corresponding to the human body rhythm. For example, the frequency range in which a human can feel a vibration phenomenon is approximately 0.1 to 500 Hz, and among these, the frequency range that can affect the human body is known to be approximately 1 to 90 Hz. In addition, it is known that human brain waves are usually approximately 10 Hz for adults. Meanwhile, most high-frequency electromagnetic waves act as noise and are more likely to have a negative effect on the body.

Also, according to the study of Lipkova and Cechak, humans emit electromagnetic energy in the frequency range of 0.5 to 30 Hz, and in particular, there are harmonic components at frequencies of 2 Hz, 3 Hz, 4.2 Hz, 16.8 Hz, and 21.3 Hz, which are related to human heart, breathing, and brain functions. (Lipkova, J. and Cechak, J. Human electromagnetic emission in the ELF band, Measurement Science Review. 5, 29-32, 2005).

When the harmonic components of the electromagnetic energy emitted from the human body resonate with the electromagnetic wave incident from the outside, the cells, tissues, and organs constituting the human body may resonate to activate bio-functions. Accordingly, the specific frequency of the electric signal output from the oscillator 120 may include at least one of 2 Hz, 3 Hz, 4.2 Hz, 16.8 Hz, and 21.3 Hz.

Meanwhile, the output module 140 may be composed of at least one of an antenna, an LED, and a speaker. For example, the output module 140 may be composed of one antenna, or may be configured to include an antenna and an LED.

When the output module 140 is configured as an antenna, the antenna may be configured as a spiral metal pattern. In this case, it is preferable that the direction in which the spiral metal pattern rotates corresponds to the spin direction of electrons that generate the longitudinal waves. For example, the spiral metal pattern may be configured as a pattern that expands while rotating clockwise from the center of the metal pattern.

The electromagnetic waves output from the output module 140 may be the longitudinal waves and have low frequencies. For example, the frequency of the longitudinal waves output from the output module 140 may include at least one of 2 Hz, 3 Hz, 4.2 Hz, 16.8 Hz, and 21.3 Hz.

When the output module 140 includes an LED, the LED is more effective in activating body functions when it is a blue LED or an orange LED.

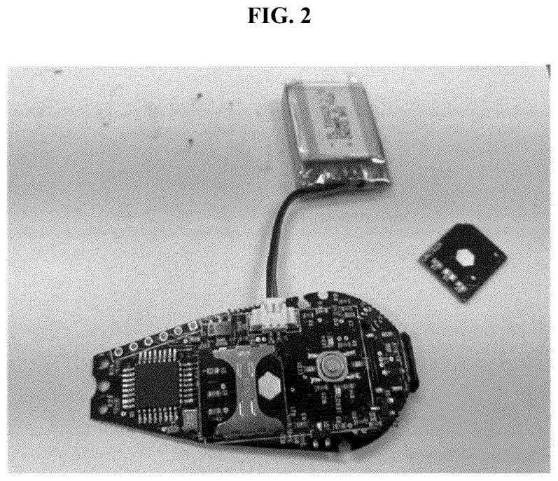

FIG. 2 is a photograph illustrating an actually manufactured product of the bio-function activation device according to the present invention.

Referring to FIG. 2, the bio-function activation device may actually be made very small and light, and thus users can easily wear the bio-function activation device without any burden.

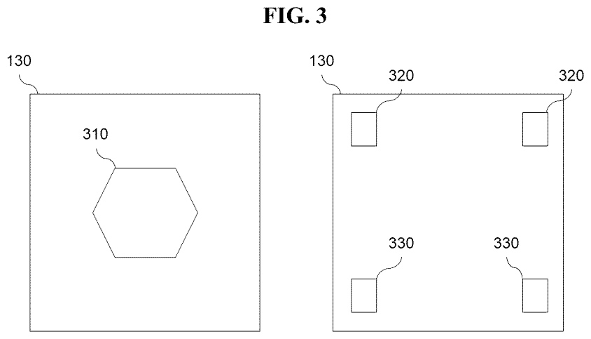

FIG. 3 is a diagram schematically illustrating the configuration of the modulation module of the bio-function activation device according to the present invention.

According to FIG. 3, the modulation module 130 according to the present invention includes an input terminal (not shown) into which the electric signal of the specific frequency is input, metal patterns 310, 320, and 330 for modulating an electromagnetic wave generated by the input electric signal of a specific frequency, and an output terminal (not shown) for outputting the electric signal that generates the electromagnetic wave modulated by the metal patterns 310, 320, and 330. Here, the electromagnetic wave modulated by the metal patterns 310, 320, and 330 may be the longitudinal wave.

The electric signal passing through the modulation module 130 is modulated according to the magnetic characteristics of the metal patterns 310, 320, and 330. In this case, the magnetic characteristics of the metal patterns 310, 320, and 330 are due to magnetic information recorded in the metal pattern, which may be information stored in advance by magnetizing the metal patterns 310, 320, and 330. In this case, the magnetic information recorded in the metal patterns 310, 320, and 330 may be information on the spin direction of electrons.

In addition, the metal patterns 310, 320, and 330 are formed in a predetermined form (shape, size, and position). This is to adjust the characteristics of the electromagnetic wave modulated by the metal patterns 310, 320, and 330. For example, the intensity or energy characteristics of the electromagnetic wave output from the output module 140 can be adjusted according to the shapes of the metal patterns 310, 320, and 330.

Since the user using the bio-function activation device 100 may be exposed to electromagnetic waves output from the bio-function activation device 100 for a long period of time, it is necessary to adjust the energy applied to the body by the electromagnetic waves to an appropriate level. For example, when the intensity of the electromagnetic waves output from the output module 140 is strong, the bio-function of the user may be activated in the short term, but the strong electromagnetic waves may cause side effects to the body of the user in the long term. Therefore, the intensity and energy characteristics of the electromagnetic waves output from the output module 140 should be adjusted so that the bio-function of the user can be appropriately adjusted without causing a burden on the body of the user even when used for a long period of time. In this case, it is preferable that the intensity of the electromagnetic waves output from the output module 140 is similar to the intensity of the electromagnetic waves emitted from the human body, and it is preferable that the energy of the electromagnetic waves has a characteristic of being uniformly distributed.

To this end, the metal patterns 310, 320, and 330 are formed differently on one surface and the other surface of the modulation module 130. The metal pattern 310 is formed in the center of one surface of the modulation module 130, and the metal patterns 320 and 330 are smaller in size than the metal pattern 310 and are formed to be spaced apart from each other on the other surface of the modulation module 130. The metal patterns 320 and 330 formed on the other surface of the modulation module 130 may be formed at positions corresponding to the positions of the input electrode and the output electrode of the modulation module 130. Here, the positions corresponding to the positions of the input electrode and the output electrode may be positions identical to the positions of the input electrode and the output electrode, positions close to the input electrode and the output electrode, or positions corresponding to a surface opposite to the surface on which the input electrode and the output electrode are formed. In addition, the plurality of metal patterns 320 and 330 formed on the other surface of the modulation module 130 may be disposed to be spaced apart at a certain distance from the center of the modulation module 130. As the metal patterns 310, 320, and 330 are formed in this manner, the energy of the electromagnetic wave output from the output module 140 may have a characteristic of being uniformly distributed.

Here, the metal patterns 310, 320, and 330 may be directly passed through or in close proximity to an electric signal passing through the modulation module 130. When the electric signal directly passes through the metal patterns 310, 320, and 330, the metal patterns 310, 320, and 330 may be directly connected to a wire through which the electric signal passes within the electromagnetic wave modulation module 130. When the electric signal passes through and in close proximity to the metal patterns 310, 320, and 330, the wire through which the electric signal passes within the electromagnetic wave modulation module 130 may be disposed in close proximity to the metal patterns 310, 320, and 330. In addition, the metal patterns 310, 320, and 330 can be directly passed through or in close proximity to the electric signal passing through the modulation module 130, respectively. For example, the metal pattern 310 formed on one surface of the modulation module 130 may be disposed so that an electric signal passes through it in close proximity, and the metal patterns 320 and 330 formed on the other surface may be disposed so that an electric signal passes through it directly. Conversely, the metal pattern 310 formed on one surface of the modulation module 130 may be disposed so that the electric signal passes through it directly, and the metal patterns 320 and 330 formed on the other surface may be disposed so that the electric signal passes through it in close proximity.

In addition, the modulation module 130 may further include an electromagnetic wave adjustment circuit (not shown) capable of adjusting the intensity and energy characteristics of the electromagnetic waves modulated by the metal patterns 310, 320, and 330. The electromagnetic wave adjustment circuit may be composed of a resistor (R) and a capacitor (C), and by adjusting the resistance value of the circuit and the capacitor capacity, the intensity and energy characteristics of electromagnetic waves modulated by the metal patterns 310, 320, and 330 may be adjusted.

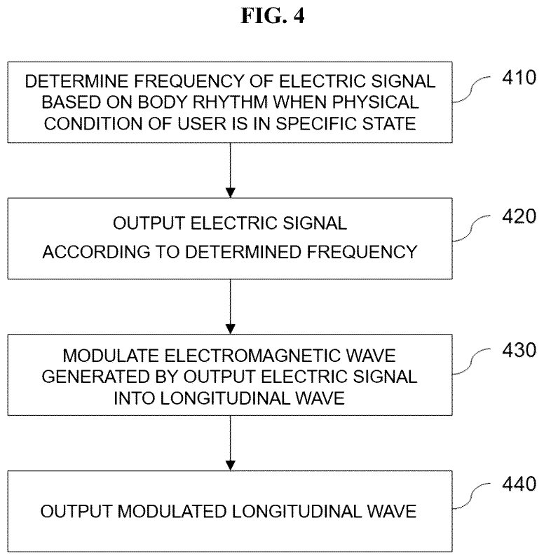

FIG. 4 is a flow chart illustrating a bio-function activation method for changing the physical condition of the user to the specific state according to the present invention.

Referring to FIG. 4, in the bio-function activation method, first, in Step 410, the frequency of the electric signal is determined based on the body rhythm when the physical condition of the user is in the specific state.

According to the determined frequency of the electric signal, if the frequency of the oscillator 120 is set, in Step 420, the oscillator 120 outputs the electric signal of the specific frequency according to the determined frequency. In this case, it is preferable that the electric signal is a square wave of positive voltage.

In Step 430, the modulation module 130 receives the electric signal output from the oscillator 120 and modulates the electromagnetic wave generated by the electric signal into the longitudinal wave.

In Step 440, the modulation module 130 outputs the electric signal in which an electromagnetic wave is modulated into the longitudinal wave, and the output module 140 receives the electric signal output from the modulation module 130 and outputs the modulated longitudinal wave according to the received electric signal to the external space.

Here, the frequencies of the electromagnetic wave generated by the electric signal output from the oscillator 120 and the modulated longitudinal wave output to the external space are determined by the frequency determined in Step 410.

In addition, in the Step 430 of modulating the electromagnetic wave generated by the output electric signal into the longitudinal wave, the intensity and energy characteristics of the longitudinal wave may be adjusted by the metal patterns 310, 320, and 330 formed in a predetermined shape for modulating an electromagnetic wave according to the electric signal.

The bio-function activation effect of the electromagnetic waves modulated according to the present invention was demonstrated through various experiments. Physical fatigue, ocular potential, and blood flow activity were tested.

Ten healthy male and female test subjects who had suffered from diseases of the nervous system in the head region were asked to use the bio-function activation device including the electromagnetic wave modulation module according to the present invention while watching digital content via an electronic device. The content was viewed for 30 minutes, and the physical fatigue, ocular potential, and blood flow activity were measured before and after use. Specifically, the physical fatigue was measured using Flicker, eye blinking was measured using the ocular potential (Electrooculogram), and blood flow activity at the corners of the eyes and the tip of the nose was measured using a thermal imaging camera. Flicker is one of the physiological measurement methods used to objectively evaluate central nervous system fatigue. The ocular potential may record potential changes due to eye blinking by utilizing the potential difference between the cornea and the retina. Facial skin temperature may be evaluated by measuring changes in the blood flow activity due to activation of the autonomic nervous system.

The results of the physical fatigue measurement showed that the difference in physical fatigue tended to decrease by an average of 1.44% after using the bio-function activation device.

TABLE 1
Result of physical fatigue T-test

| | | Paired Difference | | | |
|---|---|---|---|---|---|
| | | | Std. | | |
| Classification | Content | Mean | Deviation | t | P |
| Non-use of | Before | −0.400 | 1.635 | −1.094 | 0.288 |
| bio-function | viewing/after | | | | |
| activation device | Viewing | | | | |
| Use of | Before | −0.300 | 2.431 | −0.573 | 0.573 |
| bio-function | viewing/after | | | | |
| activation device | Viewing | | | | |

The results of eye electrophysiological measurements showed that the average blink difference decreased by 17.3% after using the bio-function activation device.

TABLE 2
Result of ocular potential T-test

| | | Paired Difference | | | |
|---|---|---|---|---|---|
| | | | Std. | | |
| Classification | Content | Mean | Deviation | t | P |
| Non-use of | Before | 6.534 | 7.233 | 2.710 | 0.027 |
| bio-function | viewing/after | | | | |
| activation device | Viewing | | | | |
| Use of | Before | 4.322 | 5.194 | 2.497 | 0.037 |
| bio-function | viewing/after | | | | |
| activation device | Viewing | | | | |

As the result of measuring the blood flow activity, the difference in blood flow activity decreased by an average of 0.45% after using the bio-function activation device, and in the case of the tip of the nose, the difference in blood flow activity tended to decrease by an average of 1.63%.

TABLE 3
Result of blood flow activity T-test

| | | Paired Difference | | | |
|---|---|---|---|---|---|
| | | | Std. | | |
| Classification | Content | Mean | Deviation | t | P |
| Non-use of | Before | −0.405 | 0.683 | −2.651 | 0.016 |
| bio-function | viewing/after | | | | |
| activation device | Viewing | | | | |
| (eye corner point) | | | | | |
| Use of | Before | −0.245 | 2.431 | −2.057 | 0.054 |
| bio-function | viewing/after | | | | |
| activation device | Viewing | | | | |
| (eye corner point) | | | | | |
| Non-use of | Before | −1.370 | 1.026 | −4.221 | 0.002 |
| bio-function | viewing/after | | | | |
| activation device | Viewing | | | | |
| (nose tip point) | | | | | |
| Use of | Before | −0.820 | 0.844 | −3.071 | 0.013 |
| bio-function | viewing/after | | | | |
| activation device | Viewing | | | | |
| (nose tip point) | | | | | |

In conclusion, it was confirmed that when using the electromagnetic waves modulated according to the present invention, the physical fatigue, ocular potential, and blood flow activity were improved. In other words, it was confirmed that bio-functions were activated.

The above-described embodiments of the present invention have been disclosed for the purpose of illustration, and those skilled in the art having common knowledge of the present invention will be able to make various modifications, changes, and additions within the spirit and scope of the present invention, and such modifications, changes, and additions should be considered to fall within the scope of the following patent claims.

What is claimed is:

1. A bio-function activation device comprising:
an oscillator that outputs an electric signal of a specific frequency, and modulates an electromagnetic wave according to the electric signal of the specific frequency to output a longitudinal wave corresponding to the specific frequency,
wherein the specific frequency is a frequency determined based on a body rhythm when a physical condition of a user is in a specific state in order to change the physical condition of the user to the specific state.

2. A bio-function activation device comprising:
a battery that supplies power;
an oscillator that is electrically connected to the battery and outputs an electric signal of a specific frequency when the power is applied from the battery;
a modulation module that is electrically connected to the oscillator, receives the electric signal of the specific frequency output from the oscillator, modulates an electromagnetic wave generated by the electric signal of the specific frequency, and outputs an electric signal in which the electromagnetic wave is modulated; and
an output module that is electrically connected to the modulation module, receives an electric signal output from the modulation module, and outputs the modulated electromagnetic wave according to the received electric signal to an external space.

3. The bio-function activation device of claim 2,
wherein the electric signal of the specific frequency output from the oscillator is for changing a physical condition of a user to a specific state, and
a frequency is determined based on a body rhythm when the physical condition of the user is in the specific state.

4. The bio-function activation device of claim 3,
wherein the specific frequency of the electric signal output from the oscillator includes at least one of 2 Hz, 3 Hz, 4.2 Hz, 16.8 Hz, and 21.3 Hz.

5. The bio-function activation device of claim 2,
wherein the electric signal of the specific frequency output from the oscillator is a square wave of positive voltage.

6. The bio-function activation device of claim 2,
wherein the modulation module includes a metal pattern formed in a predetermined shape for modulating the electromagnetic wave according to the electric signal, and
when the electric signal passes through the modulation module, the electric signal passes directly through or passes in close proximity to the metal pattern.

7. The bio-function activation device of claim 6,
wherein the metal pattern is formed in a center of one surface of the modulation module, and
a plurality of metal patterns are formed at positions corresponding to positions of an input electrode and an output electrode of the modulation module on the other surface of the modulation module.

8. The bio-function activation device of claim 7,
wherein the plurality of metal patterns formed on the other surface are disposed to be spaced apart at a certain distance from the center of the modulation module.

9. The bio-function activation device of claim 6,
wherein the modulation module adjusts intensity and energy characteristics of the electromagnetic wave output from the output module according to the predetermined shape of the metal pattern.

10. The bio-function activation device of claim 6,
wherein the modulation module modulates the electromagnetic wave according to magnetic characteristics of the metal pattern, and
the magnetic characteristics of the metal pattern are determined by magnetic information recorded in the metal pattern.

11. The bio-function activation device of claim 10,
wherein the modulation module further includes an electromagnetic wave adjustment circuit of adjusting intensity and energy characteristics of the electromagnetic wave output to the output module.

12. The bio-function activation device of claim 2,
wherein the output module includes one of an antenna, an LED, and a speaker,
the electromagnetic wave output from the output module is a longitudinal wave, and
a frequency of the electromagnetic wave output from the output module is a low frequency.

13. The bio-function activation device of claim 12,
wherein the antenna includes a spiral metal pattern for emitting the electromagnetic wave, and
the spiral metal pattern is a pattern that extends while rotating clockwise from a center of the spiral metal pattern in accordance with a spin direction of an electron that generates the longitudinal wave.

14. A bio-function activation method for changing a physical condition of a user to a specific state, the bio-function activation method comprising:
determining a frequency of an electric signal based on a body rhythm when the physical condition of the user is in the specific state;
outputting an electric signal according to the determined frequency;
modulating an electromagnetic wave generated by the output electric signal into a longitudinal wave; and
outputting the modulated longitudinal wave to an external space,
wherein the electric signal is a square wave of positive voltage, and
frequencies of the electromagnetic wave generated by the outputted electric signal and the modulated longitudinal wave output to the external space are determined by the determined frequency.

15. The bio-function activation method of claim 14,
wherein in the modulating of the electromagnetic wave generated by the output electric signal into the longitudinal wave,
intensity and energy characteristics of the longitudinal wave are adjusted by a metal pattern formed in a predetermined shape for modulating the electromagnetic wave according to the electric signal.
